(19) United States
(12) Patent Application Publication
(10) Pub. No.: US 2022/0079857 A1
(43) Pub. Date: Mar. 17, 2022

(54) PREPARATION FOR TREATING BODY ODOR

(71) Applicant: Assured Hygiene Products Ltd., Edmonton (CA)

(72) Inventor: Rae Robert Farrell, Edmonton (CA)

(21) Appl. No.: 17/472,551

(22) Filed: Sep. 10, 2021

Related U.S. Application Data
(60) Provisional application No. 63/077,900, filed on Sep. 14, 2020.

(51) Int. Cl. A61K 8/368; A61K 8/365; A01N 37/36; A01P 1/00; A61Q 15/00

(52) U.S. Cl. CPC A61K 8/368; A61K 8/365; A61Q 15/00; A01P 1/00; A01N 37/36

(57) ABSTRACT

A preparation of effective amounts of glycolic acid and salicylic acid in an aqueous solution for reducing body-odor-causing bacteria, where a concentration of glycolic acid is about 10% (w/w) or less; a concentration of the salicylic acid is about 2% (w/w) or less; and a pH of the preparation is between about 2.5-4.

### TECHNICAL FIELD

This relates to a preparation used to treat body odor, and in particular, a preparation that kills body odor-causing bacteria.

### BACKGROUND

Body odor, or bromhidrosis, may result from the interaction of sweat secreted by and bacteria carried on the skin of the body. The odor-causing bacteria, staphylococcus hominis, is a coagulase-negative member of the bacterial genus staphylococcus, consisting of gram-positive, spherical cells in clusters. It occurs commonly as a harmless commensal on human and animal skin and is known for producing thioalcohol compounds that contribute to body odor.

Typical treatments for body odor include applying deodorant or antiperspirant and frequent washing with soap and water. Other more invasive treatments may include injecting Botox to block nerve impulses to sweat glands or removing sweat glands.

### SUMMARY

According to an aspect, there is provided a preparation comprising glycolic acid and salicylic acid in an aqueous solution, comprising effective amounts for reducing body-odor-causing bacteria. The preparation may comprise a concentration of the glycolic acid of about 10% (w/w) or less; a concentration of the salicylic acid of about 2% (w/w) or less; and a pH of between about 2.5-4.

According to other aspects, the preparation may comprise about 1.5% (w/w) glycolic acid and about 0.5% (w/w) salicylic acid; the pH may be between about 3.5-3.8 or between about 2.5-3; at least a portion of the glycolic acid may be anionic.

According to an aspect, a preparation may be suitable for application to skin for the treatment of body odor, or for the treatment of surfaces.

The above features may be combined in any reasonable combination. Further aspects will be apparent from the description and claims below.

### DETAILED DESCRIPTION OF PREFERRED EMBODIMENTS

There will now be described a preparation that is able to kill staphylococcus hominis, odor-causing bacteria that is found on human skin and that may be transferred to other surfaces, such as sports equipment, footwear, furniture, etc. In general, the preparation includes effective amounts of glycolic acid and salicylic acid to reduce the amount of odor-causing bacteria. The preparation may be designed for use in different applications, such as being applied to skin, to fabrics or other substrates, etc. The intended application may involve different considerations, such as selection of a pH suitable for application to skin or sensitive surfaces, other additives, etc.

It has been found that combining glycolic acid and salicylic acid cooperate to provide beneficial results. In particular, glycolic acid, an alpha hydroxy, acts as a bacteria membrane disrupter that thins the cell walls of the bacteria, while salicylic acid, a beta hydroxy, enters a bacterial cell wall and kills the bacteria. As such, when combined, glycolic acid may make it easier for salicylic acid to pass through a bacterial cell wall.

For skin applications, salicylic acid is known to penetrate into the skins mantle, such as to clean out excess oils and dirt, while glycolic acid is unable to penetrate as deeply. By combining salicylic acid and glycolic acid, it has been found that salicylic acid helps transport glycolic acid deeper within the skin's mantle than would otherwise be the case. Once in the mantle with salicylic acid, glycolic acid weakens the bacterial cells and enhances the ability of salicylic acid to induce bacterial cell death in the staphylococcus hominis as discussed above. In this manner, salicylic acid and glycolic acid cooperate to more effectively kill bacteria on surfaces including skin, as well as within the skin's mantle.

In one example, a preparation with salicylic acid and glycolic acid may be applied to skin to reduce the amount of bacteria on the skin surface and within the skin mantle. In some cases, such a preparation may be able to prevent or reduce the amount of bacteria from returning to a significant degree for up to between 24 to 36 hours.

In another example, the solution may be used to treat a fabric or other substrate to reduce the amount of bacteria and may also prevent or reduce the amount of bacteria from returning until the solution is exhausted or removed.

A suitable formula may have up to about 2% (w/w) salicylic acid and up to about 10% (w/w) glycolic acid, although the formula may depend in part on the intended application.

The concentrations may be less than these amounts, provided that the concentrations are still effective for the intended use. The formula may have a pH of about 2.5-4, or about 3.5-3.8 such as for skin applications, or about 2.5-3 such as for surface application. In other examples, the formula may have different amounts of salicylic acid and glycolic acid in solution, provided that the end result represents an effective amount of each ingredient, in an effective combination, and in a form that is considered safe for the intended application, such as by a pH level and ingredient concentration that may be specified by regulations for these types of products.

Other ingredients in the formula may include scents, moisturizers, or other components that may improve the user-experience. The other ingredients should be selected to avoid interfering with the function of salicylic acid and glycolic acid outside an acceptable range and based on the intended application.

In one example, the preparation may be made by mixing 2% salicylic acid, 6% sodium lactate, and between 9% and 15% propylene glycol in an aqueous solution, where the sodium lactate and propylene glycol act as a solvent for the salicylic acid. This is added to a second aqueous solution in a ratio of between 1:2.5 and 1:3.5, where the second aqueous solution includes about 2.2% glycolic acid and optionally about between 0.8% and 2% d-panthenol as a moisturizer. In this example, the final solution includes about 0.5-0.6% salicylic acid and about 1.5-1.6% glycolic acid with a pH of about 3.1. The pH was adjusted up to about 3.5-3.8 for skin applications by adding a suitable amount of sodium citrate, or reduced to about 2.5-2.8 for surface applications by adding an additional amount of glycolic acid. Other additives may also be used to control the pH of the preparation. In general a preferred pH range of about 2.5-4 allows for an acceptable and effective delivery of the salicylic acid and glycolic acid.

The carrier may be designed based on the expected use and delivery method. For example, the carrier may be suitable for use in a liquid applicator, as an aerosol, or as a liquid spray. The concentration and ingredients may vary, depending on whether the solution is to be applied to fabric or other substrate, to regular skin, sensitive skin, etc. Other delivery methods and carriers may be used, depending on the preferences of the user. If intended for use on a substrate such as fabric or other material, the substrate may be treated with the preparation as a liquid during the manufacturing process and then dried. The carrier may be prepared differently whether the preparation is intended to be applied to skin or a substrate or based on the method of application.

Glycolic acid may be used in an anionic form in the preparation. Anionic surfactants possess a negative charge on their hydrophilic end that helps the surfactant molecules interact with other particles to form “bubble-like” arrangements called micelles. In a preparation as described herein, these micelles may be formed from glycolic acid and may include the bacteria being targeted, which allows them to be more effectively targeted and destroyed.

For example, in sports garment and equipment, the odor created by the staphylococcus hominis bacteria may linger after the garment or equipment are taken off or are no longer being used. The bacteria will continue to feed on other bacteria on the garment or equipment and give off a foul body odor. If not treated, the foul odor will slowly dissipate over time until there is no longer any other food source. However, the above formula may be applied to kill the staphylococcus hominis bacteria to prevent further odor production.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be one and only one of the elements.

The scope of the following claims should not be limited by the preferred embodiments set forth in the examples above and in the drawings but should be given the broadest interpretation consistent with the description as a whole.

What is claimed is:

1. A preparation comprising effective amounts of glycolic acid and salicylic acid in an aqueous solution for reducing body-odor-causing bacteria, wherein:
a concentration of the glycolic acid is about 10% (w/w) or less;
a concentration of the salicylic acid is about 2% (w/w) or less; and
the preparation has a pH of between about 2.5-4.

2. The preparation of claim 1, comprising about 1.5% (w/w) glycolic acid and about 0.5% (w/w) salicylic acid.

3. The preparation of claim 1, wherein at least a portion of the glycolic acid is anionic.

4. The preparation of claim 1-3, wherein the pH is between about 3.5-3.8.

5. The preparation of claim 1-4, wherein the pH is between about 2.5-3.

6. The use of a preparation as defined in any one of claims 1-4 for the treatment of body odor.

7. The use of a preparation as defined in any one of claim 1-3 or 5 for the treatment of surfaces.
